(19) United States
(12) Patent Application Publication
(10) Pub. No.: US 2024/0218438 A1
(43) Pub. Date: Jul. 4, 2024

(54) IMPROVEMENTS IN OR RELATING TO DIGESTION OF REACTION PRODUCTS

(71) Applicant: LumiraDx UK Ltd., London (GB)

(72) Inventors: Victor Perez, Imperial Beach, CA (US); Alexis Quintilliani, San Diego, CA (US); Bryan Kraynack, La Jolla, CA (US); Daiwei Shen, Vista, CA (US); Jarrod Provins, San Clemente, CA (US)

(21) Appl. No.: 18/563,211

(22) PCT Filed: May 27, 2022
(86) PCT No.: PCT/GB2022/051352

§ 371 Date: Nov. 21, 2023

Related U.S. Application Data
(60) Provisional application No. 63/193,649, filed on May 27, 2021.

(30) Foreign Application Priority Data
Jun. 22, 2021 (GB) 2108936.2

(51) Int. Cl. C12Q 1/6848; B01L 3/00; C12Q 1/44; C12Q 1/6806

(52) U.S. Cl. CPC C12Q 1/6848; B01L 3/5023; C12Q 1/44; C12Q 1/6806

(57) ABSTRACT

Disclosed herein is a method of causing enzymatic digestion of in vitro synthesised nucleic acid (especially DNA), the method comprising the steps of: (a) combining reagents, in the presence of a temporarily substantially inactive nuclease, to form in vitro synthesised nucleic acid; and (b) subsequently permitting or causing the substantially inactive nuclease to regain substantial nuclease activity after a period of time has elapsed sufficient to allow detection of the in vitro synthesised nucleic acid, such that the in vitro synthesised nucleic acid is digested by the nuclease.

### FIELD OF THE INVENTION

The present invention provides a method of causing enzymatic digestion of nucleic acid, and a composition useful in performing the method.

### BACKGROUND OF THE INVENTION

It is a trivial process for the person skilled in the art to bring about synthesis of nucleic acid using suitable template molecules, in the presence of nucleotide triphosphates (NTPs) and a nucleic acid polymerase enzyme in suitable reaction conditions of temperature, pH and salt concentration.

Moreover, the principles of nucleic acid synthesis reactions have been manipulated to achieve nucleic acid amplification reactions, in which multiple copies of a nucleic acid sequence of interest are synthesised. Such amplification reactions are thus able to form the basis for highly sensitive assays, in which a sequence of interest may potentially be initially present in a sample at very low copy number, but multiple copies of the sequence are produced as a result of an amplification procedure, simplifying detection of the presence of the sequence of interest.

The first nucleic acid amplification procedure to be devised was the polymerase chain reaction (PCR), and subsequently many other nucleic acid amplification techniques have been developed, the majority of which are isothermal, and thus avoid the need for thermal cycling, which is an essential requirement in PCR.

A non-exhaustive list of such isothermal amplification techniques includes: signal mediated amplification of RNA technology (“SMART”; WO 99/037805); nucleic acid sequence-based amplification (“NASBA” Compton 1991 Nature 350, 91-92); rolling circle amplification (“RCA” e.g. see Lizardi et al., 1998 Nature Genetics 19, 225-232); loop-mediated amplification (“LAMP” see Notomi et al., 2000 Nucl. Acids Res. 28, (12) e63); recombinase polymerase amplification (“RPA” see Piepenberg et al., 2006 PLoS Biology 4 (7) e204); strand displacement amplification (“SDA”); helicase-dependent amplification (“HDA” Vincent et al., 2004 EMBO Rep. 5, 795-800): transcription mediated amplification (“TMA”), single primer isothermal amplification (“SPIA” see Kurn et al., 2005 Clinical Chemistry 51, 1973-81); self-sustained sequence replication (“3SR”); and nicking enzyme amplification reaction (“NEAR”).

In ‘NEAR’ (e.g. as disclosed in US2009/0017453 and EP 2,181,196), forward and reverse primers (referred to in US 2009/0017453 and EP 2,181,196 as “templates”) hybridise to respective strands of a double stranded target and are extended. Further copies of the forward and reverse primers (present in excess) hybridise to the extension product of the opposite primer and are themselves extended, creating an “amplification duplex”. Each amplification duplex so formed comprises a nicking site towards the 5′ end of each strand, which is nicked by a nicking enzyme, allowing the synthesis of further extension products. The previously synthesised extension products can meanwhile hybridise with further copies of the complementary primers, causing the primers to be extended and thereby creating further copies of the “amplification duplex”. In this way, exponential amplification can be achieved.

Yet another amplification reaction is “STAR” (Selective Temperature Amplification Reaction). Unlike NEAR, STAR is not isothermal and involves a deliberate reduction in the temperature of the reactants from an initially elevated temperature. The reaction is disclosed and described in detail in WO 2018/002649. A quantitative variant of STAR, known as “qSTAR”, is disclosed and described in WO 2019/135074.

EP2071034 discloses a method of treating a solution in order to destroy any ribonucleic acid after performing an amplification reaction which amplifies an RNA molecule, with the aim of reducing the risk of the amplified RNA contaminating subsequent RNA amplification reactions. The method comprises performing an RNA amplification reaction in the presence of a ribonuclease and at least one ribonuclease inhibitor (such that the ribonuclease is inhibited), and subsequently inactivating the ribonuclease inhibitor to allow the ribonuclease to degrade the RNA amplification products. The technique is exemplified by the use of porcine ribonuclease inhibitor (“PRI”), which was irreversibly inactivated by heating to 65° C. for 10 minutes, permitting subsequent digestion of the amplified RNA by RNase A, which is not inactivated by such treatment.

### SUMMARY OF THE INVENTION

In a first aspect the invention provides a method of causing enzymatic digestion of in vitro synthesised nucleic acid (preferably DNA), the method comprising the steps of combining reagents, in the presence of a temporarily substantially inactive nuclease, to form in vitro synthesised nucleic acid, and subsequently permitting or causing the substantially inactive nuclease to regain substantial nuclease activity after a period of time has elapsed sufficient to allow detection of the in vitro synthesised nucleic acid, such that the in vitro synthesised nucleic acid is digested by the nuclease.

The person skilled in the art will appreciate that, in performing the method of the invention, it is not essential for the nuclease to be completely inactive during the entire period of in vitro synthesis of the nucleic acid. For example, where the nucleic acid reagents (especially the primers, templates or other oligonucleotides) necessary for the synthesis are present in great excess, some loss due to nuclease-mediated digestion may be readily tolerated. Equally, the nuclease may be completely or almost completely inactive at the start of the nucleic acid synthesis reaction, but the conditions of the reaction (such as the concentration of magnesium ions or similar divalent metal cations in the reaction mixture, for example) may be such that the nuclease becomes at least partially active during the course of the synthesis reaction.

It will be apparent that the amount of specific activity of the nuclease that can be tolerated during the nucleic acid synthesis will depend on a number of factors including the reagent concentration, the reaction conditions, the mass of nuclease present, the duration of the synthesis reaction etc. Accordingly, the following discussion represents only a guide, and the person skilled in the art can readily determine for themselves by trial and error, given the teaching of the present specification, the level of nuclease activity which can be tolerated during any particular nucleic acid synthesis reaction. In preferred embodiments, the nuclease will be at least 90% inactive at the start of the nucleic acid synthesis reaction (i.e. will possess no more than 10% of the specific activity exhibited once the nuclease enzyme has been maximally reactivated), more preferably the nuclease will be at least 95% inactive at the start of the nucleic acid synthesis reaction, and most preferably at least 99% inactive. Such nucleases may be considered to be “substantially inactive”.

Conversely, it may not be necessary for the nuclease to be completely reactivated in order to obtain the benefits of the invention. Thus, for example, a nuclease which is only 80% reactivated in terms of its maximal specific activity may be more than adequate to fully digest all of the nucleic acid synthesised in an in vitro nucleic acid synthesis reaction. Such a nuclease may be considered to be “substantially active”. Preferably, once reactivated, the nuclease will possess at least 85% of its maximal activity, more preferably at least 90%, and most preferably at least 95%. Further, whilst complete digestion of the synthesised nucleic acid may be preferred, it might not be essential, such that a nuclease which is not completely reactivated is nevertheless sufficient to greatly reduce the risk of contamination by residual nucleic acid following performance of a nucleic acid synthesis reaction in accordance with the method of the present invention.

The synthesised nucleic acid may be detected directly or indirectly, and numerous techniques for detection of nucleic acid are known to those skilled in the art and form no part of the present invention. Indirect detection methods comprise, for example, detecting a molecule or other moiety which is formed, stabilised or otherwise rendered detectable as a result of the creation and/or presence of the in vitro synthesised nucleic acid. The detection may optionally also comprise quantification of the synthesised nucleic acid.

There are a great many suitable detection and/or quantification techniques known, including: gel electrophoresis, mass spectrometry, lateral flow capture, incorporation of labelled nucleotides, intercalating or other fluorescent dyes, enzyme labels, electrochemical detection techniques, molecular beacons and other probes, especially specifically hybridising oligonucleotides or other nucleic acid-containing molecules.

Nucleic acid detection methods may employ the use of dyes that allow for the specific detection of double-stranded DNA. Intercalating dyes that exhibit enhanced fluorescence upon binding to DNA or RNA are well known. Dyes may be, for example, DNA or RNA intercalating fluorophores and may include inter alia the following: acridine orange, ethidium bromide, pico green, propidium iodide, SYBR™ I, SYBR™ II, SYBR™ gold, TOTO™-3 (a thiazole orange dimer), OliGreen™ and YOYO™ (an oxazole yellow dimer).

In a preferred embodiment, the in vitro synthesised nucleic acid is the product of a nucleic acid amplification reaction, especially a non-isothermal amplification such as a STAR amplification (e.g. as described in WO2018/002649) or a qSTAR amplification (e.g. as described in WO2019/135074), both of which require a deviation of at least 2° C. during performance of the reaction, more typically a deviation of at least 5° C. and preferably a temperature deviation in the range 5-10° C. during performance of the reaction. In the case of STAR, the temperature deviation is a temperature reduction only, whilst in qSTAR the temperature deviation comprises a shuttling between a lower temperature and an upper temperature. The person skilled in the art is very familiar with the reagents required, suitable concentrations thereof and appropriate reaction conditions (e.g. pH, temperature, buffer and salt concentrations), in order to perform in vitro nucleic acid synthesis and/or nucleic acid amplification. The in vitro synthesised nucleic acid will generally comprise or consist of DNA, and reagents necessary for this synthesis include a DNA polymerase, one or more dNTPs (preferably a mixture comprising each of dATP, dCTP, dGTP and dTTP), a template sequence (which may be RNA or DNA) which is single-stranded or rendered single-stranded, and a primer, oligonucleotide, or other nucleic acid sequence comprising a portion which can hybridise to the template and provides a free 3′ end for extension by the DNA polymerase. Alternatively, instead of separate template and primer, a hairpin oligonucleotide could be used which self-primes, thus acting as both template and primer.

There are many DNA polymerases, from prokaryotic or eukaryotic sources, known to those skilled in the art. In principle, any of these may be utilised in the method of the invention. The various DNA polymerases are generally well characterised and have different properties and characteristics (e.g. different temperature stabilities; presence or absence of exonuclease proof reading ability) and the person skilled in the art is well used to selecting and appropriate DNA polymerase according to the DNA to be performed and the associated reaction conditions.

In particular, the method of the present invention is especially advantageous when the nucleic acid is synthesised as part of an assay process for detection and/or quantification of a nucleic acid sequence of interest in a sample of biological material, e.g. the diagnostic detection of an infectious agent (including, but not limited to, bacteria, viruses, fungi and protozoa) in a biological sample from a human or animal subject. The sample may, for instance, comprise or consist of the following: whole blood, plasma, serum, sputum, saliva, lachrymal fluid, semen, vaginal fluid, urine or faeces. Alternatively, the sample may be an environmental sample, such as a water sample, and used for determining the microbiological quality of the water. Advantageously, the method of the invention is performed, wholly or at least in part, using an assay device such as a lateral flow or microfluidic device, especially a point-of-care diagnostic test device, a nucleic acid synthesis reaction being performed in or on such a diagnostic test device. By way of explanation, in the method of the invention, the synthesised nucleic acid, (and/or nucleic acid initially present in the sample from the subject), after detection, will be at least partly or, more preferably, wholly digested by the nuclease following its reactivation. In this way, the digestion of the nucleic acid after the assay (including any detection step) has been completed, will greatly reduce the risk of any equipment being contaminated with amplifiable fragments of nucleic acid, especially where the equipment is to be re-used in subsequent nucleic acid amplification reactions; such amplifiable fragments might otherwise lead to false positive results.

Typically the “period of time” which elapses prior to the nuclease regaining substantial activity (measured from commencement of the nucleic acid synthesis reaction) is from 1 to 48 hours, preferably 1 to 12 hours, more preferably 1 to 6 hours, and most preferably 1-2 hours. If it is desired to decrease the time which elapses between completing the in vitro nucleic acid synthesis reaction, and digestion of the nucleic acid in the reaction mixture, it is of course possible to commence reactivation of the nuclease before starting the nucleic acid synthesis reaction. For example, the nuclease could be mixed with Mg2+ ions (as described below) prior to addition of the nuclease to the reaction mixture.

The nuclease-mediated digestion of the synthesised nucleic acid can be readily confirmed by, for example, analysis of the reaction mixture, once the nuclease has regained activity. For present purposes, the synthesised nucleic acid may be considered completely digested by the nuclease if no polynucleotide longer than 15 nucleotides is detectable by electrophoretic analysis on a polyacrylamide gel pre-stained with appropriate SYBR gold II molecules. After the DNA synthesis reaction has been performed, the assay reaction mixture (or any assay device on, or in, which the reaction has been performed) may be left in suitable conditions to permit, and preferably facilitate, the digestion of the nucleic acid. This may simply involve, for example, leaving the reaction mixture (or assay device, as the case may be) at room temperature.

The nuclease used in the method of the invention may be any suitable nuclease, including an exonuclease. Preferably however the nuclease is an endonuclease, and in most embodiments will comprise a sugar non-specific endonuclease (as described below; i.e. will act on both RNA and DNA substrates). In some embodiments, the endonuclease is selected from the group consisting of heat-labile salt activated nuclease (HL-SAN, ArcticZymes, P/N70910-202, described in U.S. Pat. No. 9,422,595), HL-dsDNase (ArcticZymes, P/N 70800-201, a double stranded DNA-specific endonuclease), Cyanase™ Nuclease (Sigma Aldrich, P/N 1000), Benzonase™ Nuclease (Sigma Aldrich, P/N E8263, an endonuclease from Serratia marcescens, expressed in E. coli, and provided in ultra pure form [>99%, as judged by SDS PAGE]), Cryonase™ Cold-active nuclease (Takara, P/N 2670A, is a recombinant endonuclease originally derived from the psychrophilic organism, Shewanella sp., and purified by expression from E. coli), and OmniCleave™ endonuclease (Lucigen, P/N OC7850K).

The foregoing nucleases are all commercially available from the sources indicated. Most of the foregoing endonucleases (except HL-dsDNase) are classified as “sugar non-specific endonucleases” that require magnesium or manganese for activity (Rangarajan and Shankar, 2001 FEMS Microbiol. Rev. 25, 583-613). These endonucleases will cleave both DNA and RNA. HL-dsDNase is classified as a “sugar specific endonuclease” (i.e. it will act on DNA substrates only, not RNA), and has a high affinity specifically for double-stranded DNA. However, all the aforementioned endonucleases require magnesium or manganese for activity and would be expected to be active while in the presence of these cofactors. The inventors have found that low concentrations of Mg²⁺ ions (<0.25 mM) are desirable to delay the activation of the nuclease for an extended period. Thus, for example, with Cyanase™ nuclease and Cryonase™ cold-active nuclease, which typically carry over up to 0.25 mM Mg²⁺ ions from their suppliers' storage buffers, the enzymes exhibit essentially no reactivation after 48 hours (at a temperature of 25° C.) in the absence of additional Mg²⁺ ions.

It will be apparent however to those skilled in the art that nucleic acid synthesis reactions generally require the presence of Mg²⁺ ions, or a similar aqueous divalent metal cation, since these are essential cofactors for most polymerases used in nucleic acid synthesis. Thus, whilst the nuclease may preferably be stored (in a dry or freeze-dried state) in the absence of Mg²⁺ ions, commencing the nucleic acid synthesis reaction (in the presence of the temporarily inactivated nuclease) will require the addition of a buffer or the like comprising Mg²⁺ ions, such that the temporarily inactivated nuclease will start to be reactivated.

In a preferred embodiment the nuclease is temporarily inactivated by a process which comprises contacting the nuclease with a physiological reducing agent, such as dithiothreitol (DTT), Tris (2-carboxyethyl) phosphine (TCEP), or DTBA (dithiobutylamine). Typically the physiological reducing agent will be present at a concentration in the range 0.5-10 mM, preferably 1-10 mM, and more preferably 1-5 mM. Generally, a suitable buffer (e.g. TE) at a pH in the range 7.5-8.5 will also be present.

The preferred treatment required to cause reversible inactivation of the endonuclease will depend on the identity of the endonuclease concerned.

For example, HL-SAN and HL-dsDNase (both from ArcticZymes), are described by the manufacturer as being irreversibly inactivated by the presence of 1 mM DTT (see U.S. Pat. No. 9,422,595 and manufacturer's website). In particular U.S. Pat. No. 9,422,595 states that the enzyme (IHL-SAN) is “substantially (irreversibly) inactivated when incubated at 30° C. for 15 minutes in the presence of 10 mM DTT”; and “substantially (irreversibly) inactivated when incubated at 4° C. for 6 hours in the presence of either 10 mM DTT or 10 mM TCEP”. In fact, surprisingly and contrary to the teaching in the art, the present inventors have found that both HL-SAN and HL-dsDNase can be reversibly inactivated by treatment with a physiological reducing agent, such as 1-10 mM DTT or TCEP. This reversible inactivation can be brought about without heating (e.g. by holding at 4° C. for 6 hours in the presence of either 10 mM DTT or TCEP). Alternatively, the reversible inactivation of HL-SAN or HL dsDNase can be accomplished very quickly by heating either enzyme in the presence of a physiological reducing agent (e.g. 1-10 mM DTT or TCEP). The inventors have found that essentially complete reversible inactivation can be accomplished by incubating at 60° C. for 30 minutes. Such heating is to be preferred, since physiological reducing agents such as DTT can be rather unstable, especially at low concentration—a prolonged incubation at low temperature might therefore permit the reducing agent to become exhausted, allowing the nuclease to escape complete (reversible) inactivation and/or to become reactivated. This risk is ameliorated by a rapid incubation at elevated temperature.

Generally speaking, the other endonucleases mentioned above (Cyanase™ Benzonase™, Cryonase™ and OmniCleave™ are preferably reversibly inactivated by a process which comprises heating the enzymes in the presence of a physiological reducing agent. Conveniently the physiological reducing agent comprises DTT or TCEP, typically at a concentration in the range 1-10 mM. Advantageously, the heating comprises incubating the enzymes at 60° C. for 30 minutes which, in the presence of the physiological reducing agent, the inventors have found causes substantially complete reversible inactivation. It will be apparent to those skilled in the art that, generally, a temperature lower than 60° C. may be used if the duration of the incubation is increased.

For example, a temperature in the range 25-55° C. may conveniently be employed in some embodiments, more preferably a temperature in the range 35-55° C. In some circumstances, a temperature of 25° C. represents a substantially ambient temperature, such that heating is not necessary to achieve temporary inactivation of the nuclease, whilst in other environments heat will be required to achieve a temperature of 25° C. Heating will normally be required to attain temperatures above 30° C., and especially above 35° C. The temperature used may typically depend, at least in part, on the length of time for which the endonuclease is held at the selected temperature. Thus, for example, holding the endonuclease at 25° C. for a week (or longer) might be sufficient to bring about temporary inactivation. Temperatures above 60° C. are generally to be avoided, as they tend to cause irreversible inactivation of at least some of the endonuclease.

Once the nuclease has been reversibly inactivated, it may be desirable to keep the enzyme substantially inactivated at least until the in vitro synthesis of the nucleic acid has commenced, and possibly until the in vitro synthesised nucleic acid has been detected. Maintaining the nuclease in substantially (reversibly) inactivated form can be accomplished or facilitated in a number of ways, including, but not limited to, one or more of the following: (a) keeping the inactivated nuclease at low temperature (i.e. 4° C. or below), more preferably freezing the nuclease (e.g. typically at −20° C. or below); (b) air drying or freeze-drying the inactivated nuclease; (c) keeping the inactivated nuclease in an environment where the concentration of Mg²⁺ or Mn²⁺ ions (co-factors of the nuclease) is at a level at which the enzyme remains substantially inactive—preferably the concentration of available Mg²⁺ or Mn²⁺ ions is less than 0.25 mM, more preferably at or below 0.1 mM. Conveniently (a) and (c), or (b) and (c), may be adopted in combination. The reversibly inactivated nuclease may be stored in a vessel, such as an Eppendorf® tube, or may be stored on a solid support (such as a lateral flow test strip or on a microfluidic test device). In other embodiments, the reversibly inactivated endonuclease may be incorporated into a “master mix”, which is subsequently used, in diluted form, in performing a nucleic acid reaction such as a DNA amplification reaction.

The amount of nuclease desired to be present in the reaction mixture may depend on the amount of nucleic acid to be digested, the extent of digestion desired (complete or partial), the amount of time required to elapse before the nucleic acid is to be digested, the temperature and other conditions (such as pH, salt concentration etc). Typically the temporarily inactivated nuclease may be present in the reaction mixture at a concentration in the range 0.1 Units/μL to 500 Units/μL, more typically in the range 0.2 Units/μL to 250 Units/μL.

The deliberate incorporation of a nuclease into a nucleic acid synthesis reaction mixture during nucleic acid synthesis is counter-intuitive, because the presence of a nuclease in such a reaction would normally be expected to digest the nucleic acid synthesis primers (prior to, and/or after, hybridisation to complimentary template, depending on the characteristics of the nuclease) thus adversely affecting the reaction. However, in the method of the present invention, inactivation of certain nucleases, e.g. by subjecting them to contact with a physiological reducing agent such as TCEP (i.e. tris(2-carboxyethyl)phosphine) or a dithio compound, such as DTT (i.e. dithiothreitol) or DTBA (dithiobutylamine), (and typically also incubation at a temperature in the range 25-60° C.) is temporary and reversible, so that it is possible to permit the presence of a nuclease in a nucleic acid synthesis reaction mixture, especially an amplification reaction mixture, during active nucleic acid synthesis, the nuclease being initially inactivated, but regaining activity over time, allowing the nucleic acid synthesis reaction to proceed substantially unimpeded for long enough to permit detection of the synthesised nucleic acid.

It is known to use an enzyme such as a nuclease to eliminate or reduce contamination of nucleic acid-containing samples, or reagents and buffers and the like to be used in nucleic acid reactions, but such conventional approaches contact the sample/reagent/buffer etc, as the case may be, with a nuclease enzyme before performing any amplification step, and require that the enzyme is either removed or permanently and irreversibly inactivated before conducting the amplification procedure. Typically the enzymes used in this way are DNA repair enzymes such as Uracil N-glycosidase (“UNG”), or an endonuclease such as DNase I, which enzymes are heat-labile and irreversibly inactivated by heating to high temperature (e.g. 95° C. for 5 minutes or longer).

It is an advantage of the present invention that, incorporation of the reversibly inactivated nuclease into a DNA amplification reaction mixture, as an integral component thereof, prior to performing a DNA amplification, allows the amplification to be preformed in e.g. a sealed vessel or on a solid support (e.g. in a microfluidic test device) and that, subsequently, the nuclease will become reactivated without requiring any further input from the user to degrade the amplification products or other nucleic acid present, and thereby reduce the risk of contamination when performing subsequent reactions.

In a second aspect the invention provides a composition of use in performing the method of the first aspect of the invention, the composition comprising a DNA polymerase, at least one dNTP, and a temporarily substantially inactive nuclease in reactivatable form. Preferably the nuclease used in the composition of the invention is an endonuclease, specifically an endonuclease which can act on DNA substrates. In some embodiments, the endonuclease is a sugar non-specific endonuclease. In preferred embodiments, the endonuclease is selected from the group consisting of heat-labile salt activated nuclease (HL-SAN, ArcticZymes, P/N70910-202), HL-dsDNase (ArcticZymes, P/N 70800-201), Cyanase™ Nuclease (Sigma Aldrich, P/N 1000), Benzonase™ Nuclease (Sigma Aldrich, P/N E8263), Cryonase™ Cold-active nuclease (Takanara, P/N 2670A), and OmniCleave™ endonuclease (Lucigen, P/N OC7850K). As described above, the nuclease is preferably rendered temporarily substantially inactive by a process which comprises contacting the nuclease with a physiological reducing agent, such as dithiothreitol (DTT), Tris (2-carboxyethyl) phosphine (TCEP), or DTBA (dithiobutylamine). Suitable conditions for temporarily inactivating the nuclease include contacting the nuclease with a 1-10 mM concentration (preferably 1-5 mM) of the reducing agent, normally in a suitable buffer (such as TE) at a pH in the range 7.5-8.5 or so. Other suitable conditions will be apparent to those skilled in the art or can be determined without inventive effort with the benefit of the present disclosure. In particular, heating may preferably be included to assist in temporary inactivation of the nuclease: a temperature in the range 25-60° C. may typically be employed in some embodiments for this purpose.

The composition will typically comprise a plurality of NTPs, preferably a mixture comprising each of dATP, dCTP, dGTP and dTTP. Alternatively, or additionally, the composition (and the method) of the invention may involve the use of one or more variant NTPs (e.g. uracil), or an analogue of an NTP which can be incorporated into an in vitro synthesised nucleic acid by a DNA polymerase. Numerous NTP analogues are known to those skilled in the art. Alternatively, or additionally, the composition may comprise a DNA polymerase.

The composition of the invention may be provided as a plurality of aliquots. Typically the aliquots will be essentially identical, each aliquot being provided in a separate vessel or container such as an Eppendorf® tube or the like. The composition of the invention may be provided, for example, in liquid form, frozen, dried (e.g. air-dried or freeze-dried) or in other solid form suitable for solution in water, distilled water, aqueous buffer etc.

In other embodiments, the composition of the invention may be provided on a solid support, such as lateral flow test strip, or on a microfluidic test device: in such embodiments, the composition may be provided as a liquid (e.g. in a reservoir or the like), or may be provided in dry form, possibly releasably immobilised on the solid support. The composition of the invention may additionally comprise other components, such one or more buffers, one or more salts (especially Mg salts), preservatives, cryoprotectants (e.g. trehalose or the like) etc.

Thus in a third aspect, the invention provides a test device for testing for the presence and/or amount of a nucleic acid sequence of interest in a sample, the test device comprising one or more reagents for performing an in vitro nucleic acid synthesis reaction, (preferably a DNA amplification reaction) and a temporarily substantially inactive nuclease in reactivatable form. It will be appreciated that the substantially inactive nuclease is allowed to regain substantial nuclease activity after a period of time has elapsed sufficient to allow detection of the in vitro synthesised nucleic acid, such that the in vitro synthesised nucleic acid is digested by the nuclease. The test device may advantageously be one which may subsequently be reused in a further test. The test device may comprise a composition in accordance with the second aspect of the invention, as defined above. The test device may also optionally comprise one or more reagents for detecting the presence of the in vitro synthesised nucleic acid, such as a molecular beacon, labelled probe or the like.

The invention will now be further described by way of illustrative example and with reference to the accompanying drawings, in which:

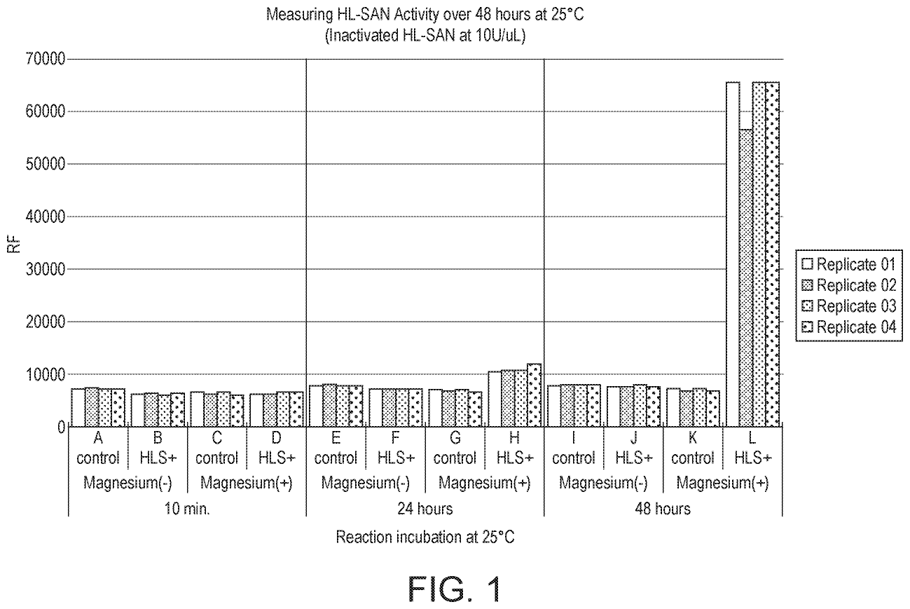

FIG. 1 is a bar chart showing the endonuclease activity of heat-labile salt-activated nuclease (HL-SAN), after storage for different time periods at 25° C. in the presence or absence of Mg²⁺ ions;

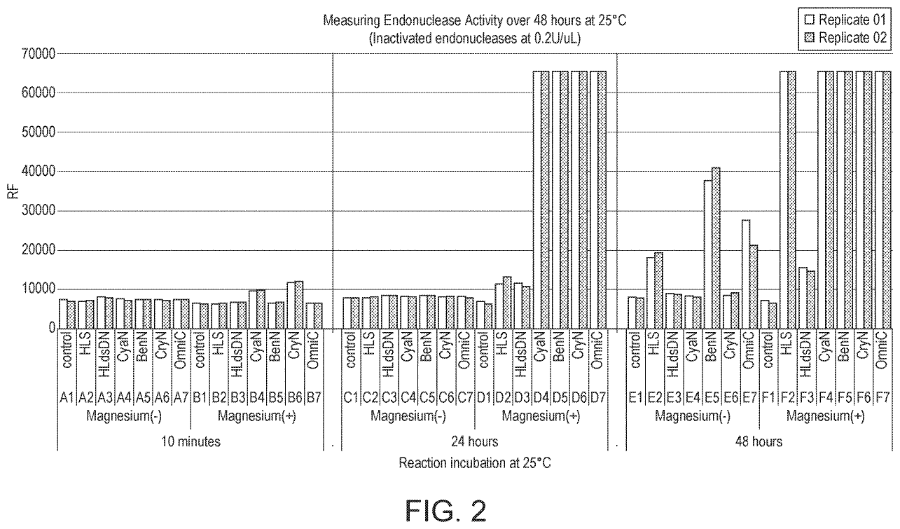

FIG. 2 is a bar chart showing the endonuclease activity of a variety of different endonucleases after storage for different time periods at 25° C. in the presence or absence of Mg²⁺ ions;

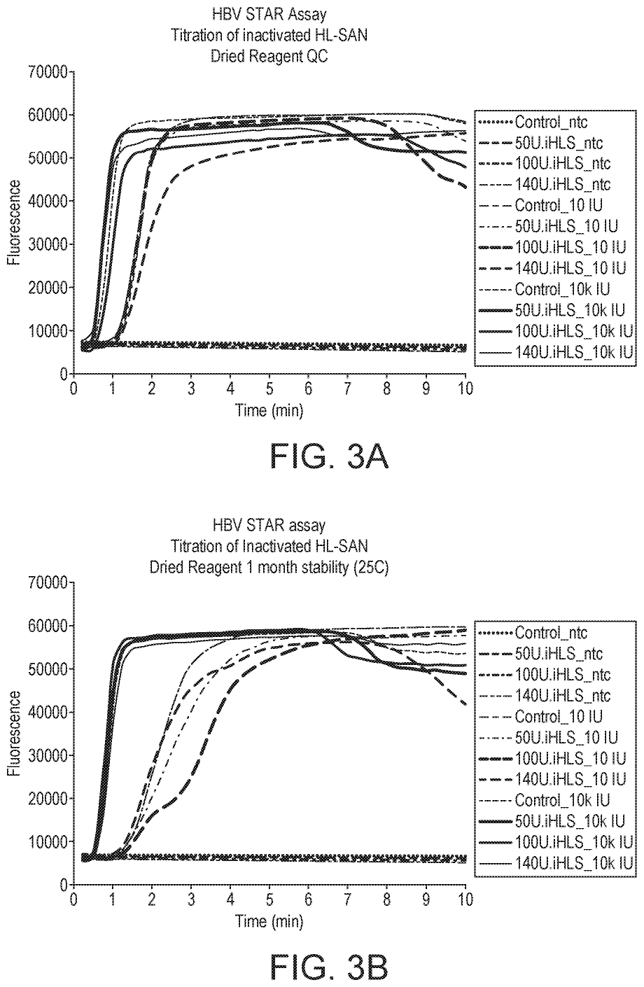

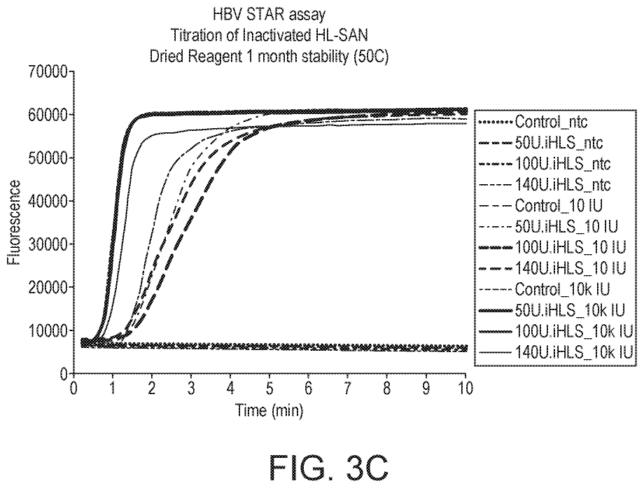

FIGS. 3A-3C are graphs of Relative Fluorescence (Arbitrary units) against time (minutes), showing the amount of fluorescence generated (indicative of the amount of nucleic acid amplification) by performing STAR nucleic acid amplification reactions using reagents, including heat-labile salt activated nuclease (HL-SAN), without prior storage (FIG. 3A) or after storage for 1 month at 25° C. (FIG. 3B) or 50° C. (FIG. 3C); and

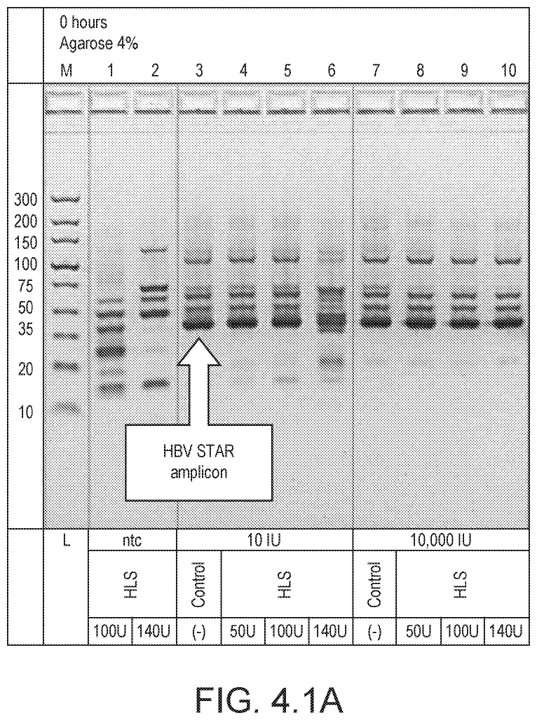

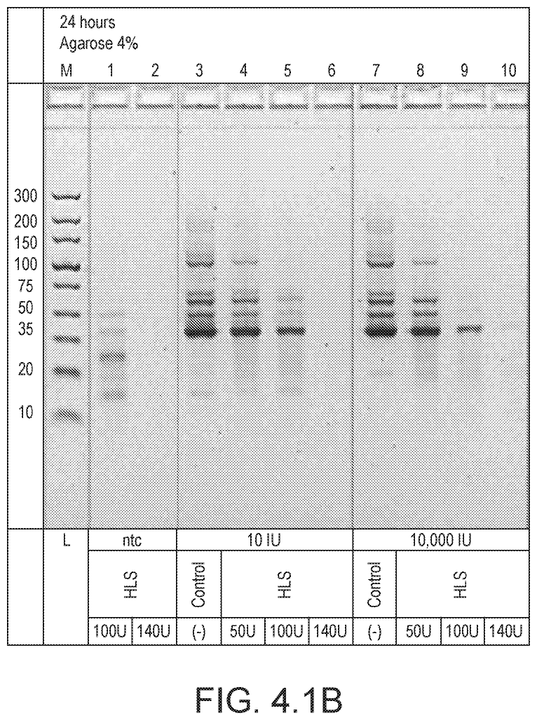

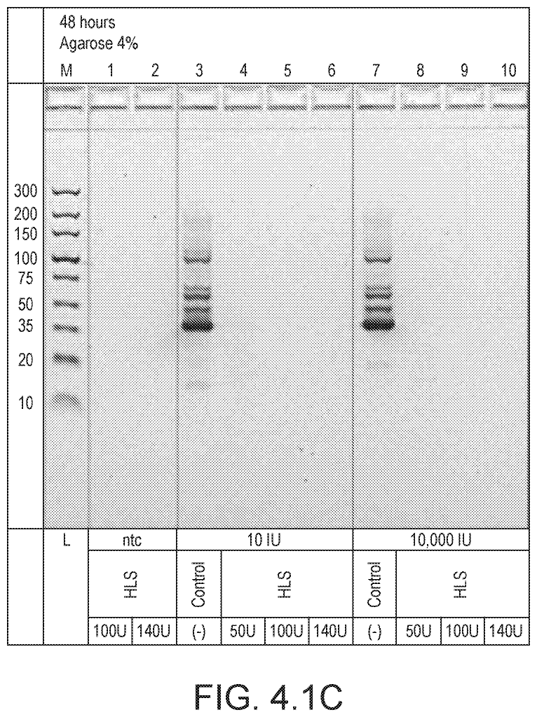

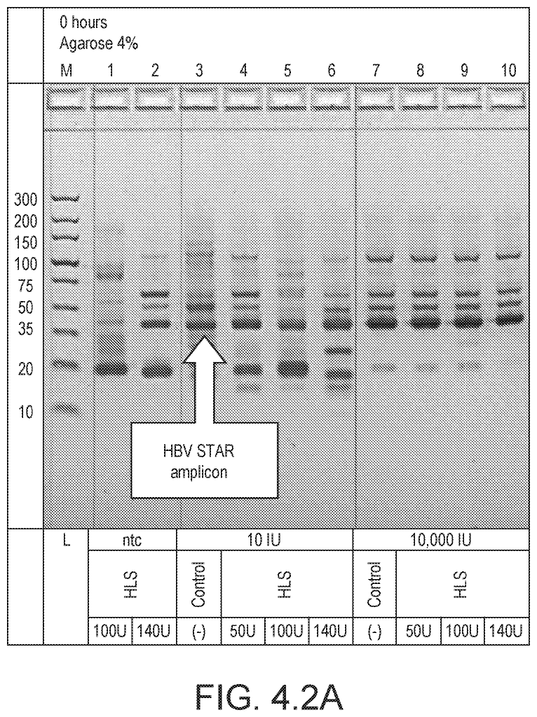

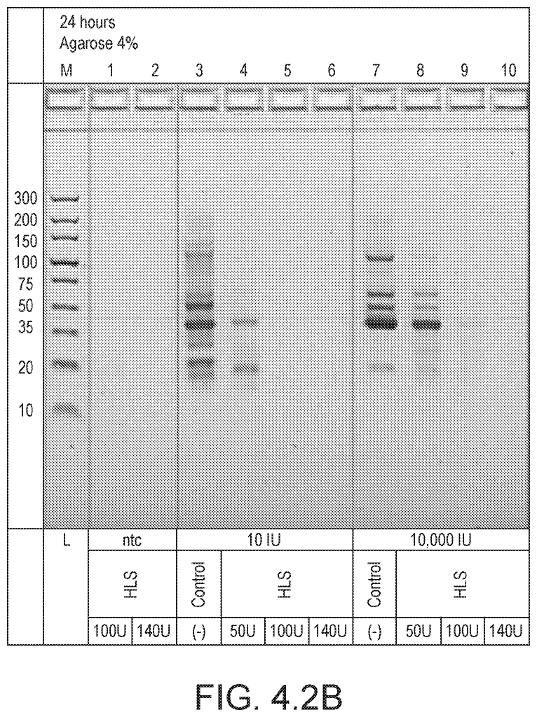

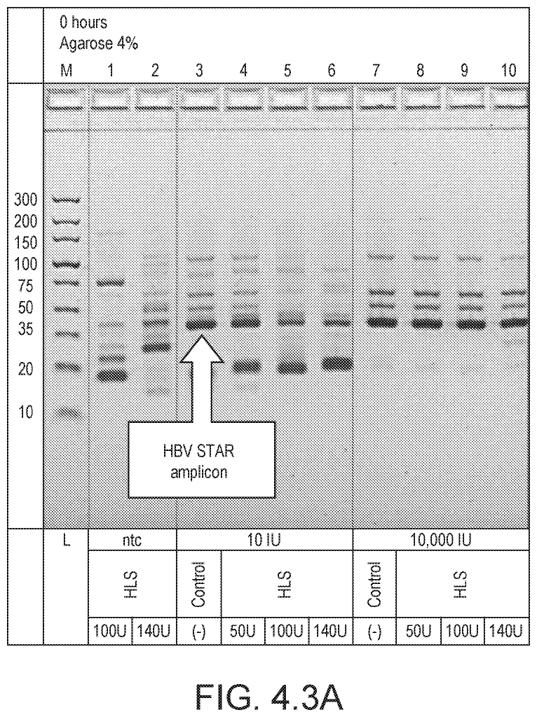

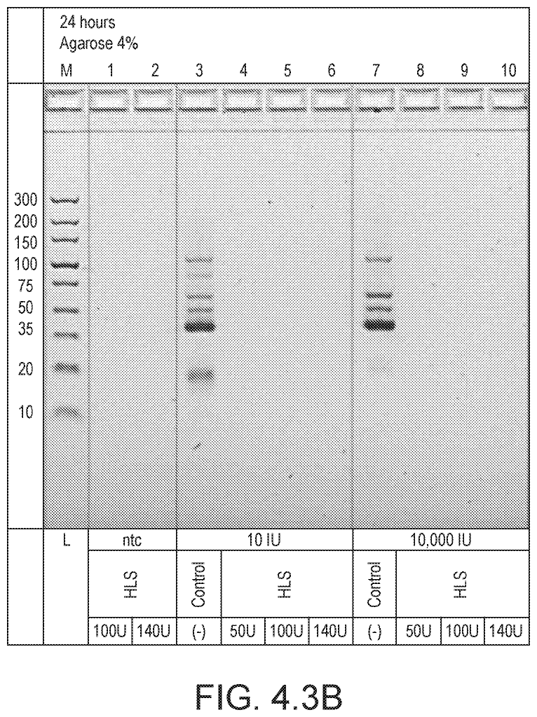

FIGS. 4.1 (a-c), 4.2 (a,b) and 4.3 (a,b) are images of agarose gels loaded with amplification reaction products with or without exposure to reactivated HL-SAN endonuclease.

### EXAMPLES

This invention relates to the utilization of nuclease for the digestion of nucleic acid amplification assay products. In the examples below, the endonuclease was incorporated into the assay without adversely affecting performance of the assay, and acted as a safeguard against contamination by digesting nucleic acid, including amplified products, within 24 to 48 hours of assay completion.

The temporary inactivation of the nuclease involved exposure to a physiological reducing agent and, optionally, elevated temperature. Once temporarily inactivated, the nuclease could be incorporated into an amplification assay mix such as, e.g. a STAR assay master mix (see Example 3 below), and subsequently dried down at room temperature. The STAR assay was run without the nuclease affecting assay performance. Upon completion of the assay run, the reactions were left at room temperature and analyzed using agarose gel electrophoresis to confirm endonuclease reactivation. The endonuclease reactivated over time as the reducing agent lost its ability to break-up disulfide bonds (due to atmospheric oxidation of the reducing agent), allowing the nuclease to return to its active form. A major advantage of this method is that the nuclease reactivation rate can be manipulated by adjusting protein concentrations, adjusting reducing agent concentrations, and/or altering storage conditions.

### Example 1: Reversible Inactivation of HL-SAN

Heat-labile Salt-Activated Nuclease (HL-SAN) was combined with 1 mM reducing agent, tris(2-carboxyethyl)phosphine (TCEP), in a Tris-EDTA (TE) buffer (pH8). Alternatively, a dithio-compound, such as dithiothreitol (DTT) can be used as the reducing agent. HL-SAN stock solution was at a concentration of 615 U/μL (ArcticZymes, Batch no: RD1742-c, glycerol-free, magnesium free), and diluted to 200 U/μL for inactivation. HL-SAN was inactivated by incubation at 60° C. for 30 minutes. Inactivated HL-SAN was equilibrated to room temperature for 10 minutes. Inactivated HL-SAN was stored at 40° C. for 1 hour prior to testing. An in-house assay (Dual-labeled Probe Digestion Assay, or “DLP-DA”) was used to measure inactivation and reactivation.

The DLP-DA comprises a dual-labeled probe (DLP) with a 5′-fluorophore and a 3′-quencher in a specified buffer. The DLP is designed with complementary DNA sequences on the 5′ and 3′ ends so that a long double-stranded DNA stem is formed as the complementary regions hybridize to one another. When the stem is closed the fluorophore is suppressed by the proximity of the quencher. In the presence of an endonuclease, the double-stranded DNA stem is digested and destabilized. Ultimately, the fluorophore is separated from the quencher and a fluorescent signal is generated. HL-SAN was tested at 10 U/μL with the DLP-DA in the absence and presence of magnesium ions. Reactions were incubated at room temperature at various time points, and end-point fluorescence was measured on a Stratagene MX3005P instrument. The results are shown in FIG. 1.

Nuclease activity was compared between reactions either with inactivated HL-SAN (HLS+) or without (control). Four reaction replicates were incubated at 25° C. for each condition, and the raw fluorescence (RF) was analyzed at several time points. Increasing fluorescence was directly related to increasing nuclease activity. In FIG. 1, the different shading of the columns represents the different replicates of the experiment, darker shades representing results for replicates 1 and 2, and lighter shades showing results for replicates 3 and 4.

HL-SAN remained inactive in the absence of Mg²⁺ ions after 10 minutes at 25° C. (FIG. 1 columns A, B): fluorescence values for the control (<9,500 RF) (FIG. 1 col. A) were similar to those of the inactivated HL-SAN (<9,500 RF) (FIG. 1 col. B). HL-SAN remained inactive in the presence of magnesium after 10 minutes at 25° C. (FIG. 1 cols. C, D). Fluorescence values for the control (<9,500 RF) (FIG. 1 col. C) were similar to those of the inactivated HL-SAN (<9,500 RF) (FIG. 1 col. D).

HL-SAN remained inactive in the absence of Mg²⁺ ions after 24 hours at 25° C. (FIG. 1 columns E, F). Fluorescence values for the control (<9,500 RF) (FIG. 1 col. E) were similar to those of the inactivated HL-SAN (<9,500 RF) (FIG. 1 col. F). Low-level nuclease activity was observed in the presence of magnesium after 24 hours at 25° C. (FIG. 1 cols. G, H). Elevated fluorescent values were observed for the inactivated HL-SAN (>10,000 RF) (FIG. 1 col. H).

HL-SAN remained inactive in the absence of Mg²⁺ ions after 48 hours at 25° C. (FIG. 1 cols. I, J). Fluorescence values for the control (<9,500 RF) (FIG. 1 col. I) were similar to those of the inactivated HL-SAN (<9,500 RF) (FIG. 1 col. J). Robust nuclease activity was observed in the presence of Mg²⁺ ions after 48 hours at 25° C. (FIG. 1 cols. K, L). Maximum fluorescence values were observed for the inactivated HL-SAN (>55,000 RF) (FIG. 1 col. L).

The data showed that the method for inactivating HL-SAN was effective. HL-SAN remained inactive in the absence of Mg²⁺ ions over a period of up to 48 hours at 25° C. This (delayed) reactivation occurred only in the presence of Mg²⁺ ions. This characteristic makes HL-SAN a suitable endonuclease to be incorporated into e.g. a STAR assay formulation. For prolonged inactivation, it is important that the endonuclease storage buffer is substantially free of Mg²⁺ ions (e.g. below 0.1 mM, more preferably below 0.05 mM), and preferably substantially free of similar divalent metal ions, such as manganese ions.

### Example 2: Reversible Inactivation of Various Endonucleases

It was decided to test endonucleases other than HL-SAN to see if they too could be reversibly inactivated.

All endonucleases were normalized to 2 U/μL for this experiment. All the endonucleases were provided in a 50% glycerol containing buffer with 1-5 mM magnesium. HL-SAN stock was at a concentration of 25.4 U/μL (ArcticZymes, P/N 70910-202). HL-dsDNase stock was at a concentration of 2 U/μL (ArcticZymes, P/N 70800-201). Cyanase™ nuclease stock was at a concentration of 50 U/μL (Ribosolutions, P/N 1000). Benzonase™ nuclease stock was at a concentration of >250 U/μL (Sigma Aldrich, P/N E8263). Cryonase™ cold-active nuclease stock was at a concentration of 20 U/μL (Takanara, P/N 2670A). OmniCleave™ endonuclease stock was at a concentration of 200 U/μL (Lucigen, P/N OC7850K). Inactivation steps were identical to the method previously described (Example 1 above). Endonucleases were diluted to 1 U/μL for inactivation. The methods for measuring nuclease inactivation and reactivation were identical to those described above for HL-SAN. Endonucleases were tested at 0.2 U/μL using the DLP-DA in the absence and presence of Mg²⁺ ions.

The results are shown in FIG. 2.

Nuclease activity was compared between reactions with an inactivated endonuclease and without (control). Two reaction replicates were incubated at 25° C. for each condition, and the raw fluorescence was analyzed at several time points. Inactivated endonucleases are represented on the bar graph with the following nomenclature:
Control: No endonuclease
HLS: HL-SAN (ArcticZymes, P/N 70910-202)
HLdsDN: HL-dsDNase (ArcticZymes, P/N 70800-201)
CyaN: Cyanase™ nuclease (Ribosolutions, P/N 1000)
BenN: Benzonase™ nuclease (Sigma Aldrich, P/N E8263)
CryN: Cryonase™ cold-active nuclease (Takanara, P/N 2670A)
OmniC: OmniCleave™ endonuclease (Lucigen, P/N OC7850K)

Endonucleases remained inactive in the substantial absence of Mg²⁺ ions after 10 minutes at 25° C. (FIG. 2 cols. A1-A7). Fluorescence values for the control (<9,500 RF) (FIG. 2 col. A1) were similar to those of all of the inactivated endonucleases (<9,500 RF) (FIG. 2 cols. A2-A7).

Very low-level nuclease activity was observed for some endonucleases (Cyanase™ & Cryonase™ cols. B4, B6) in the presence of Mg²⁺ after 10 minutes at 25° C., whilst fluorescence values for the control (<9,500 RF) (FIG. 2 col. B1) were similar to the other four of the inactivated endonucleases (<9,500 RF) (FIG. 2 cols. B2, B3, B5, B7).

All endonucleases remained essentially inactive in the absence of Mg²⁺ after 24 hours at 25° C. (FIG. 2 cols. C1-C7). Fluorescence values for the control (<9,500 RF) (FIG. 2 col. C1) were similar to those of all of the inactivated endonucleases (<9,500 RF) (FIG. 2 cols. C2-C7). Nuclease activity was observed in the presence of magnesium after 24 hours at 25° C. (FIG. 2 cols. D1-D7). Slightly elevated fluorescent values were observed for two of the inactivated endonucleases (>9,500 RF) (FIG. 2 cols. D2, D3), whilst maximum fluorescence values were observed for four of the inactivated endonucleases (>55,000 RF) (FIG. 2 cols. D4-D7).

Nuclease activity was measured for the endonucleases in the absence of Mg²⁺ ions after 48 hours at 25° C. (FIG. 2 cols. E1-E7). Fluorescence values for the control reactions (<9,500 RF) (FIG. 2 col. E1) were similar to three of the inactivated endonucleases (<9,500 RF) (FIG. 2 cols. E3, E4, E6). Elevated fluorescence values were observed for three of the inactivated endonucleases (>15,000 RF) (FIG. 2 cols. E2, E5, E7). Nuclease activity was observed in the presence of Mg²⁺ ions after 48 hours at 25° C. (FIG. 2 cols. F1-F7). Elevated fluorescence values were observed for one of the inactivated endonucleases (>10,000 RF) (FIG. 2 col. F3), whilst maximum fluorescence values were observed for the rest of the inactivated endonucleases (>55,000 RF) (FIG. 2 cols. F2, F4-F7).

The data showed that the method for temporarily inactivating HL-SAN can also be applied to other endonucleases. All the endonucleases were reactivated in the presence of magnesium ions after initial inactivation. However, it is important to note that magnesium ions were present in most of the endonuclease storage buffers, which makes it difficult to sustain inactivation for a long period of time. Inactivation was sustained more effectively for 48 hours at 25° C. when HL-SAN was provided in a magnesium-free buffer (FIG. 1 cols. B, F, J).

### Example 3: STAR Assay Performance with Incorporated Endonuclease

This example involves the performance of STAR (“Selective Temperature Amplification Reaction”), which is a nucleic acid amplification technique described in WO 2018/002649. STAR assay for the detection of Hepatitis B viral DNA was chosen for this study, but in principle the method of the invention can be used in conjunction with any known DNA amplification reaction, and any amplicon. The region of the HBV genome selected as the target for amplification has been previously submitted to Genbank, Accession number MN047437 (Koyaweda et al., Int. J. Infect. Dis. 90 (2020) 138-144), and appropriate STAR assay primers were designed accordingly. 10 μl STAR master mix contained the following reagents; 15 mM MgSO₄, 90 mM Tris-HCl (pH 8.5), 300 μM each dNTPs, 15 mM (NH₄)₂SO₄, 15 mM Na₂SO₄, 1 mM DTT, 0.01% Triton X-100, 7 U nicking endonuclease, and 48 U polymerase. Dried down STAR assay master mix was rehydrated with a salt mixture. Tris-EDTA pH 8.0 (TE) was added to No Target Control (NTC) wells. HBV target was added to target wells (10 IU, 10,000 IU). HBV STAR assay amplification was initiated, and real-time data was collected. HBV STAR assay dried reagents were stored at 25° C. and 50° C., then subsequently tested after 1 month. The results are shown in FIGS. 3A-3C.

FIG. 3A:

This plot illustrates the real-time amplification curves generated by the HBV STAR assay dried reagents upon completion of the drying process. For FIG. 3A, the color gradient (dark, medium, lighter, lightest) for the plot lines represents increasing amounts (Control 0 U, 50 U, 100 U, 140 U) of HL-SAN per reaction. Assay performance was compared in the presence (FIG. 3A dashed-dotted lines, solid lines) or absence (FIG. 3A dotted lines) of HBV target. F or the negative target control reactions (FIG. 3A dotted lines), no difference was observed between the control (FIG. 3A dark dotted line) and those with inactivated HL-SAN (FIG. 3A dashed-dotted lines, solid lines). All reactions showed no amplification.

In relation to the 10 IU target reactions (FIG. 3A dashed lines), no difference was observed between the control (FIG. 3A dark solid line) and those with inactivated HL-SAN (FIG. 3A medium, lighter, lightest solid lines). All reactions showed similar amplification curves.

For the 10,000 IU target reactions (FIG. 3A solid lines), no difference was observed between the control (FIG. 3A black solid line) and those with inactivated HL-SAN (FIG. 3A medium, lighter, lightest solid lines). All reactions showed similar amplification curves.

These results revealed that there was no difference in the performance of the HBV STAR assay dried reagents. The presence of the inactivated HL-SAN had no effect on assay performance.

FIG. 3B:

This plot illustrates the real-time amplification curves generated by the HBV STAR assay dried reagents that were stored at 25° C. for 1 month. This plot illustrates the real-time amplification curves generated by the HBV STAR assay dried reagents that were stored at 25° C. for 1 month. For FIG. 3B, the color gradient (dark, medium, lighter, lightest) for the plot lines represents increasing amounts (Control 0 U, 50 U, 100 U, 140 U) of HL-SAN per reaction. Assay performance was compared in the presence (FIG. 3B dashed-dotted lines, solid lines) or absence (FIG. 3B dotted lines) of HBV target.

For the negative target control reactions (FIG. 3B dotted lines), no difference was observed between the control (FIG. 3B dark dotted line) and those with inactivated HL-SAN (FIG. 3B medium, lighter, lightest dotted lines). All reactions showed no amplification.

In relation to the 10 IU target reactions (FIG. 3B dashed-dotted lines), no difference was observed between the control (FIG. 3B dark dashed-dotted line) and those with inactivated HL-SAN (FIG. 3B medium, lighter and lightest dashed-dotted lines). All reactions showed similar amplification curves.

For the 10,000 IU target reactions (FIG. 3B solid lines), no difference was observed between the control (FIG. 3B dark solid line) and those with inactivated HL-SAN (FIG. 3B medium, lighter, lightest solid lines). All reactions showed similar amplification curves.

FIG. 3C:

This plot illustrates the real-time amplification curves generated by the HBV STAR assay dried reagents that were stored at 50° C. for 1 month. For FIG. 3C, the color gradient (dark, medium, lighter, lightest) for the plot lines represents increasing amounts (Control 0 U, 50 U, 100 U, 140 U respectively) of HL-SAN per reaction. Assay performance was compared in the presence (FIG. 3C dashed-dotted lines, solid lines) or absence (FIG. 3C dotted lines) of HBV target.

For the negative target control reactions (FIG. 3C dotted lines), no difference was observed between the control (FIG. 3C dark dotted line) and those with inactivated HL-SAN (FIG. 3C medium, lighter, and lightest dotted lines). All reactions showed no amplification.

In relation to the 10 IU target reactions (FIG. 3C dashed-dotted lines), no difference was observed between the control (FIG. 3C dark dashed-dotted line) and those with inactivated HL-SAN at 50 U (FIG. 3C medium dashed-dotted line). Reactions with HL-SAN at 100 U and 140 U (FIG. 3C lighter and lightest dashed-dotted lines respectively) generated elevated fluorescence when compared to the control (FIG. 3C dark dashed-dotted line).

For the 10,000 IU target reactions (FIG. 3C solid lines), no difference was observed between the control (FIG. 3.C dark solid line) and those with inactivated HL-SAN at 50 U and 100 U (FIG. 3C medium, lighter solid lines respectively). Reactions with HL-SAN at 140 U (FIG. 3C lightest solid line) generated elevated fluorescence when compared to the control (FIG. 3C dark solid line).

These results revealed that storage at 50° C. of the HBV STAR assay dried reagents generated elevated fluorescence with incorporated HL-SAN at 100 U and 140 U. The presence of the inactivated HL-SAN had no effect on assay performance.

### Example 4 Agarose Gel Analysis of Digested STAR Assay Products

Upon completion of the HBV STAR assay test, reactions were stored at room temperature for subsequent agarose gel analysis (reactions from FIGS. 3A, 3B, and 3C).

Reactions were diluted and loaded onto Invitrogen 4% EX E-gels (Invitrogen P/N G401004). Electrophoresis executed on the Invitrogen Power-Snap E-gel system (Invitrogen P/N G8100). Images were captured/stored using the Invitrogen Power-Snap camera (Invitrogen P/N G8200). Image colour was inverted for better resolution of amplified products. HBV STAR assay reactions were tested at 0 hours (same day as test), and after 24 hours and 48 hours. The results are shown in FIGS. 4.1-4.3.

FIG. 4.1:

The 3 gel images (FIG. 4.1a, 4.1b, 4.1c) illustrate the reactivation of HL-SAN over a period of 24 and 48 hours at 25° C. HL-SAN reactivation is represented by those reactions that showed a significant loss of amplified products (HBV STAR amplicon).

HBV STAR assay dried reagents were tested with and without target (FIG. 3.1). HBV STAR assay reactions were diluted and loaded onto 4% EX E-gel upon assay completion (FIG. 4.1a).

The following legend describes the reactions loaded onto the gels:
Lane M: Ultra-low range DNA ladder (Invitrogen P/N 10488096).
Lanes 1-2: No target control (ntc) reactions with HL-SAN at 100 U and 140 U, respectively.
Lane 3: Control reaction (no endonuclease) with 10 IU of HBV target.
Lanes 4-6: Reactions with 10 IU of HBV target with incorporated HL-SAN at 50 U, 100 U, and 140 U, respectively.
Lane 7: Control reaction (no endonuclease) with 10,000 IU of HBV target
Lanes 8-10: Reactions with 10,000 IU of HBV target with incorporated HL-SAN at 50 U, 100 U, and 140 U, respectively.

At 0 hours (FIG. 4.1a), as expected, only non-specific products were observed in the NTC reactions (FIG. 4.1a Lanes 1-2). Amplified products in reactions with HL-SAN (FIG. 4.1a Lanes 4-6,8-10) showed similar bands to the control reactions (FIG. 4.1a Lanes 3,7).

At 24 hours (FIG. 4.1b), a measurable loss in amplified products was observed in the NTC reaction with HL-SAN at 100 U (FIG. 4.1b Lane 1). The NTC reaction with HL-SAN at 140 U (FIG. 4.1b Lane 2) showed a complete loss of amplified products. Target reactions with HL-SAN at 50 U and 100 U (FIG. 4.1b Lanes 4,5,8,9) showed a measurable loss of amplified products when compared to the controls (FIG. 4.1b Lanes 3,7). Target reactions with HL-SAN at 140 U (FIG. 4.1b Lanes 6,10) showed a complete loss of amplified products.

At 48 hours (FIG. 4.1c), a complete loss in amplified products was observed for all reactions with HL-SAN (FIG. 4.1c Lanes 1-2, 4-6, 8-10). Nuclease reactivation was observed after 24 hours in reactions with 140 U HL-SAN incorporated into the HBV STAR assay dried reagents. Lower concentrations, 50 U and 100 U, required 48 hours for complete nuclease reactivation.

FIG. 4.2:

The two gel images (FIG. 4.2a, 4.2b) illustrate the reactivation of HL-SAN over a period of 24 hours at 25° C. HL-SAN reactivation is represented by those reactions that showed a significant loss of amplified products (HBV STAR amplicon).

HBV STAR assay dried reagents were stored at 25° C. for 1 month and tested with and without target (FIG. 4.2).

HBV STAR assay reactions were diluted and loaded onto a 4% EX E-gel upon assay completion (FIG. 4.2a).

The following legend describes the reactions loaded onto the gels:
Lane M: Ultra-low range DNA ladder (Invitrogen P/N 10488096).
Lanes 1-2: No target control (ntc) reactions with HL-SAN at 100 U and 140 U, respectively.
Lane 3: Control reaction (no endonuclease) with 10 IU of HBV target.
Lanes 4-6: Reactions with 10 IU of HBV target with incorporated HL-SAN at 50 U, 100 U, and 140 U, respectively.
Lane 7: Control reaction (no endonuclease) with 10,000 IU of HBV target.
Lanes 8-10: Reactions with 10,000 IU of HBV target with incorporated HL-SAN at 50 U, 100 U, and 140 U respectively

At 0 hours (FIG. 4.2a), as expected, only non-specific products were observed in the NTC reactions (FIG. 4.2a Lanes 1-2). Amplified products in reactions with HL-SAN (FIG. 4.2a Lanes 4-6,8-10) showed similar bands to the control reactions (FIG. 4.2a Lanes 3,7).

At 24 hours (FIG. 4.2b), a complete loss in amplified products was observed in the NTC reactions with HL-SAN at 100 U and 140 U (FIG. 4.2b Lanes 1,2). Target reactions with HL-SAN at 50 U (FIG. 4.2b Lanes 4,8) showed a measurable loss of amplified products when compared to the controls (FIG. 4.2b Lanes 3,7). Target reactions with HL-SAN at 100 U and 140 U (FIG. 4.2b Lanes 5,6,10) showed a complete loss of amplified products.

Nuclease reactivation was observed after 24 hours in reactions with 100 U and 140 U HL-SAN incorporated into the HBV STAR assay dried reagents. At 50 U, more than 24 hours is required for complete nuclease reactivation. Dried reagents stored at 25° C. for 1 month showed an increase in HL-SAN reactivation rate for 100 U when compared to the initial test run (FIG. 4.1).

FIG. 4.3:

The two gel images (FIG. 4.3a, 4.3b) illustrate the reactivation of HL-SAN over a period of 24 hours at 25° C. HL-SAN reactivation is represented by those reactions that showed a significant loss of amplified products (HBV STAR amplicon).

HBV STAR assay dried reagents were stored at 50° C. for 1 month and tested with and without target (FIG. 3.3). HBV STAR assay reactions were diluted and loaded onto a 4% EX E-gel upon assay completion (FIG. 4.3a).

The following legend describes the reactions loaded onto the gels:
Lane M: Ultra-low range DNA ladder (Invitrogen P/N 10488096).
Lanes 1-2: No target control (ntc) reactions with HL-SAN at 100 U and 140 U, respectively.
Lane 3: Control reaction (no endonuclease) with 10 IU of HBV target.
Lanes 4-6: Reactions with 10 IU of HBV target with incorporated HL-SAN at 50 U, 100 U, and 140 U, respectively.
Lane 7: Control reaction (no endonuclease) with 10,000 IU of HBV target.
Lanes 8-10: Reactions with 10,000 IU of HBV target with incorporated HL-SAN at 50 U, 100 U, and 140 U, respectively.

At 0 hours (FIG. 4.3a), as expected, only non-specific products were observed in the NTC reactions (FIG. 4.3a Lanes 1-2). Amplified products in reactions with HL-SAN (FIG. 4.3a Lanes 4-6,8-10) showed similar bands to the control reactions (FIG. 4.3a Lanes 3,7).

At 24 hours (FIG. 4.3b), a complete loss in amplified products was observed for all reactions with HL-SAN (FIG. 4.3b Lanes 1-2, 4-6, 8-10).

Nuclease reactivation was observed after 24 hours in reactions with HL-SAN incorporated into the HBV STAR assay dried reagents. Dried reagents stored at 50° C. for 1 month showed an increase in HL-SAN reactivation rate for all HL-SAN concentrations when compared to the initial test run (FIG. 4.1). The experiment also showed that HL-SAN could digest the amplification products after their detection, and that incorporation of HL-SAN into the amplification reaction did not prevent the amplification reaction from occurring.

1. A method of causing enzymatic digestion of in vitro synthesised nucleic acid, the method comprising the steps of (a) combining reagents, in the presence of a temporarily substantially inactive nuclease, to form in vitro synthesised nucleic acid; and (b) subsequently permitting or causing the substantially inactive nuclease to regain substantial nuclease activity after a period of time has elapsed sufficient to allow detection of the in vitro synthesised nucleic acid, such that the in vitro synthesised nucleic acid is digested by the nuclease.

2. The method according to claim 1, wherein the nuclease is an exonuclease or endonuclease which acts on DNA substrates.

3. The method according to claim 1 or 2, wherein the nuclease requires the presence of aqueous magnesium and/or manganese ions for nuclease activity.

4. The method according to any one of the preceding claims, wherein the nuclease is selected from the group consisting of heat-labile salt-activated nuclease HL-SAN; HL-dsDNase; Cyanase nuclease; Benzonase nuclease; Cryonase cold-active nuclease; and OmniCleave endonuclease.

5. The method according to any one of the preceding claims, wherein the in vitro nucleic acid synthesis is synthesised as the product of a DNA amplification reaction.

6. The method according to claim 5, wherein the nucleic acid amplification reaction is an isothermal or non-thermal cycling amplification.

7. The method according to any one of the preceding claims, wherein the nuclease is temporarily substantially inactivated by a process which comprises contacting the nuclease with a physiological reducing agent.

8. The method according to claim 7, wherein the physiological reducing agent comprises dithiothreitol (DTT) or tris (2-carboxyethyl)phosphine (TCEP).

9. The method according to any one of the preceding claims, wherein the nuclease is heat-labile and the nuclease is temporarily inactivated by a process comprising incubating the nuclease at a temperature in the range 25-60° C. for at least 15 minutes.

10. The method according to any one of the preceding claims, wherein the step of permitting or causing the substantially inactive nuclease to regain substantial nuclease activity comprises contacting the nuclease with aqueous magnesium and/or manganese ions.

11. The method according to claim 10, comprising contacting the nuclease with aqueous magnesium and/or manganese ions in the concentration range 10 mM to 100 mM to facilitate reactivation of the nuclease.

12. A method of performing an in vitro DNA synthesis reaction, the method comprising the steps of: forming a DNA synthesis reaction mixture which comprises all the reactants necessary to perform the DNA synthesis reaction, the reaction mixture further comprising a DNA nuclease which is initially substantially inactive but in a reactivatable form; performing the DNA synthesis reaction; and permitting or causing the DNA nuclease to regain substantial nuclease activity so as to degrade the products of the DNA synthesis reaction.

13. A method according to claim 12, wherein the in vitro DNA synthesis reaction comprises a DNA amplification reaction.

14. A method according to claim 12 or 13, wherein one or more products of the DNA synthesis reaction are detected, directly or indirectly, prior to their digestion by the reactivated nuclease.

15. A method according to any one of claims 12-14, and further in accordance with any one of claims 1-11.

16. A composition of use in performing the method of any one of the preceding claims, the composition comprising a DNA polymerase, at least one dNTP, and a temporarily substantially reversibly inactivated nuclease in reactivatable form.

17. The composition according to claim 16, comprising a plurality of NTPs, preferably a mixture comprising each of dATP, dCTP, dGTP and dTTP.

18. The composition according to claim 16 or 17, wherein the nuclease is an exonuclease or endonuclease which acts on DNA substrates.

19. The composition according to claim 18, wherein the nuclease is selected from the group consisting of heat-labile salt activated nuclease HL-SAN; HL-dsDNase; Cyanase nuclease; Benzonase nuclease; Cryonase cold-active nuclease; and OmniCleave endonuclease.

20. The composition according to any one of claims 16-19, wherein the composition is provided as a plurality of substantially identical aliquots, each aliquot being provided in a separate vessel or container.

21. The composition according to claim 20, wherein the aliquots are provided in dried form, a frozen form, or a freeze-dried form.

22. A test device for testing for the presence and/or amount of a nucleic acid sequence of interest in a sample, the test device comprising one or more reagents for performing an in vitro nucleic acid synthesis reaction, and a temporarily substantially inactive nuclease in reactivatable form.

23. A test device according to claim 22, comprising a composition in accordance with any one of claims 16-21.

24. A lateral flow or microfluidic test device according to claim 22 or 23.
